(19) United States
(12) Patent Application Publication
(10) Pub. No.: US 2012/0226176 A1
(43) Pub. Date: Sep. 6, 2012

(54) DEVICES AND METHODS FOR RECEIVING PHYSIOLOGICAL SIGNALS

(75) Inventors: Chia-Hsien LI, New Taipei City (TW); Chu-Chia TSAI, New Taipei City (TW); Chu-Shun CHO, New Taipei City (TW); Wen-Chin WU, New Taipei City (TW); Brian-Chua CHONG, New Taipei City (TW)

(73) Assignee: WISTRON CORP., New Taipei City (TW)

(21) Appl. No.: 13/354,998

(22) Filed: Jan. 20, 2012

(30) Foreign Application Priority Data
Mar. 2, 2011 (TW) 100106844

(51) Int. Cl. A61B 5/0404; A61B 5/00

(52) U.S. Cl. 600/509; 600/300

(57) ABSTRACT

An electronic device for receiving a physiological signal of a user comprises a physiological signal receiving device and a computing device. The physiological signal receiving device comprises: a sensor, configured to receive the physiological signal of the user; a first processor, coupled to the sensor, and configured to convert the physiological signal into a digital signal; a first communication interface, configured to output the digital signal; a battery and power management system; and an ExpressCard connector, coupled to the first processor. The computing device comprises a second communication interface, configured to receive the digital signal; and an ExpressCard socket.

### CROSS REFERENCE TO RELATED APPLICATIONS

This Application claims priority of Taiwan Patent Application No. 100106844 filed on Mar. 2, 2011, the entirety of which is incorporated by reference herein.

### BACKGROUND OF THE INVENTION

1. Field of the Invention

The disclosure generally relates to a physiological signal receiving device, and more particularly relates to a physiological signal receiving device comprising an ExpressCard interface.

2. Description of the Related Art

With the progress of medical science, human beings are paying more attention to their health. However, modern people usually spend little time to have checkups due to their busy lives. A heartbeat, and one's breath and brain waves are all important indexes of health. A combination of the above indexes is helpful for people and family doctors to understand physical conditions of a person.

Traditional sensing systems for heartbeat, breath and brain waves are wired sensing systems. For example, a patient must put many electrodes on his body when using an electrocardiogram (ECG). The electrodes conduct heartbeat signals, and a device processes the heartbeat signals and then displays processed signals on a monitor. Use of wires and electrodes limit the motion of patients when in use and may make them reluctant to have checkups.

Medical devices are expensive and complex, so most people are not capable of easily examining their own physiological signals. In summary, it is necessary to invent a wireless, small and cheap physiological signal receiving device for receiving physiological signals of a person.

An ExpressCard interface is an expansion interface for information devices. The ExpressCard interface is developed by PCMCIA and supports hot swapping.

### BRIEF SUMMARY OF THE INVENTION

In one exemplary embodiment, the disclosure is directed to an electronic device for receiving a physiological signal of a user, comprising: a physiological signal receiving device, comprising: a sensor, configured to receive the physiological signal of the user; a first processor, coupled to the sensor, and configured to convert the physiological signal into a digital signal; a first communication interface, configured to output the digital signal; a battery and power management system; an ExpressCard connector, coupled to the first processor; and a computing device, comprising: a second communication interface, configured to receive the digital signal; and an ExpressCard socket.

In another exemplary embodiment, the disclosure is directed to a physiological signal receiving device for receiving a physiological signal of a user, comprising: a sensor, configured to receive the physiological signal of the user; a processor, coupled to the sensor, and configured to convert the physiological signal into a digital signal; a communication interface, configured to output the digital signal; a battery and power management system; and an ExpressCard connector, coupled to the processor.

In one exemplary embodiment, the disclosure is directed to a physiological signal receiving method for receiving a physiological signal of a user, comprising: receiving the physiological signal of the user via a physiological signal receiving device; converting the physiological signal into a digital signal via the physiological signal receiving device; outputting the digital signal wirelessly via the physiological signal receiving device; receiving the digital signal wirelessly via a computing device; and generating user status information according to the digital signal via the computing device, wherein the physiological signal receiving device comprises an ExpressCard interface.

In another exemplary embodiment, the disclosure is directed to a computing device for computing a digital signal, comprising: a processor, generating user status information according to the digital signal.

### BRIEF DESCRIPTION OF DRAWINGS

The invention can be more fully understood by reading the subsequent detailed description and examples with references made to the accompanying drawings, wherein:

FIG. 1 is a diagram illustrating an electronic device according to an embodiment of the invention;

FIG. 2 is an outlook drawing illustrating a physiological signal receiving device according to an embodiment of the invention;

FIG. 3 is a diagram illustrating a physiological signal receiving device adapted to a USB connector according to an embodiment of the invention;

FIG. 4 is a flow chart illustrating a physiological signal receiving method according to an embodiment of the invention.

### DETAILED DESCRIPTION OF THE INVENTION

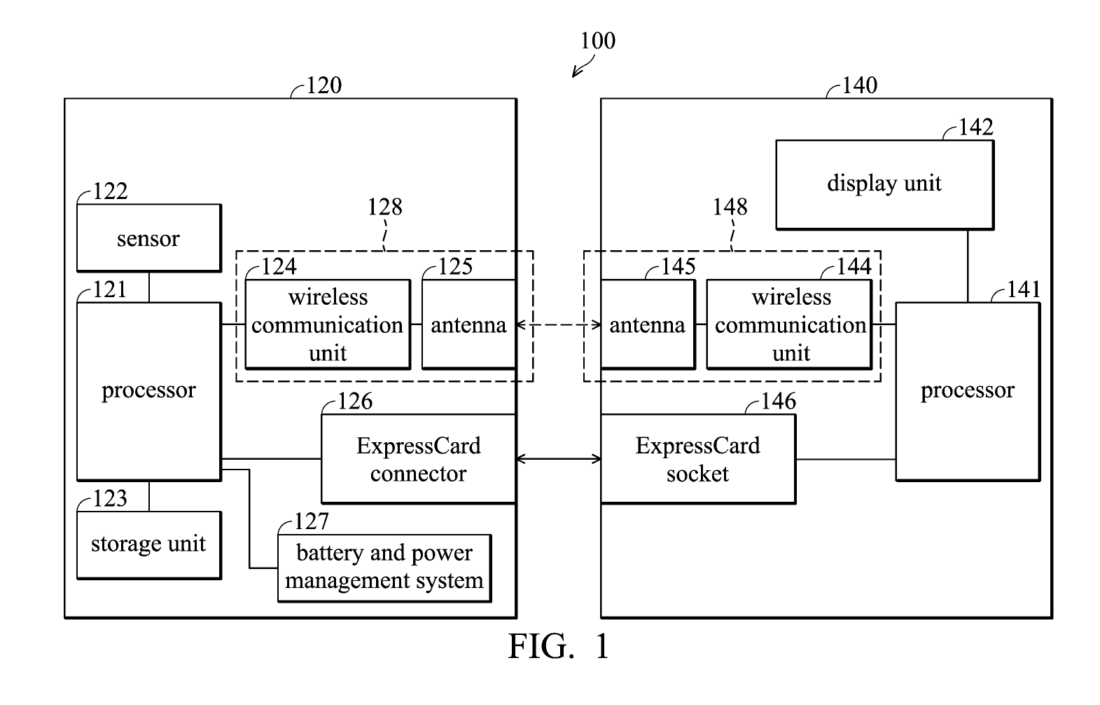

FIG. 1 is a diagram illustrating an electronic device 100 according to an embodiment of the invention. As shown in FIG. 1, the electronic device 100 comprises a physiological signal receiving device 120 and a computing device 140. The physiological signal receiving device 120 may comprise an ExpressCard interface. The computing device 140 may be a notebook, a personal computer (PC), a tablet PC or other computers that can be electrically connected to the physiological signal receiving device 120. If the computing device 140 is a notebook, the physiological signal receiving device 120 can be connected to an ExpressCard connector of the notebook and incorporated into the notebook. It is convenient to move the notebook with the physiological signal receiving device 120.

In one embodiment of the invention, as shown in FIG. 1, the physiological signal receiving device 120 comprises: a processor 121, a sensor 122, a storage unit 123, a wireless communication unit 124, an antenna 125, an ExpressCard connector 126, and a battery and power management system 127. It is noted that only necessary elements directly related to the invention are shown in FIG. 1. Other elements, such as an upper case and a lower case of the physiological signal receiving device 120, are well-known for a person of ordinary skill in the art, so they are not shown in FIG. 1. The processor 121 is electrically connected to the sensor 122, the storage unit 123, the wireless communication unit 124, the ExpressCard connector 126, and the battery and power management system 127. The antenna 125 is electrically connected to the wireless communication unit 124. The processor 121 may be an electrocardiogram chip (ECG chip), a brain wave chip or other chips utilized for converting a physiological signal, such as heartbeat, brain waves and breathe, into a digital signal. The sensor 122 may be a sensing metal or an electrode to receive a physiological signal from human body. The storage unit 123 may be a memory or a hard disc to store a physiological signal or a digital signal. The antenna 125 may be a PIFA antenna (planer inverted F antenna), a monopole antenna, a loop antenna, a helical antenna, or a chip antenna. The wireless communication unit 124 and the antenna 125 constitute a communication interface 128 that may be a wireless communication module, such as Bluetooth module, to wirelessly output a digital signal that is processed via the processor 121. In another embodiment of the invention, the communication interface 128 may be replaced with a USB (Universal Serial Bus) interface to output a digital signal that is processed via the processor 121.

As shown in FIG. 1, in another embodiment of the invention, the computing device 140 comprises: a processor 141, a display unit 142, a wireless communication unit 144, an antenna 145, and an ExpressCard socket 146. The processor 141 is electrically connected to the display unit 142, the wireless communication unit 144, and the ExpressCard socket 146. The antenna 145 is electrically connected to the wireless communication unit 144. The display unit 142 may be a television, a computer monitor, or a projector. The wireless communication unit 144 and the antenna 145 constitute a communication interface 148 that may be a wireless communication module, such as a Bluetooth module, to wirelessly receive a digital signal and then transmit the digital signal to the processor 141. In another embodiment of the invention, the communication interface 148 may be replaced with a USB interface to receive a digital signal and then transmit the digital signal to the processor 141.

After receiving a digital signal, the processor 141 may display the digital signal on the display unit 142 directly, or calculate user status information according to the digital signal and then display the user status information on the display unit 142. In one embodiment, the physiological signal is a heartbeat or pulse, the digital signal is an electrocardiogram, and the user status information is a sentiment index that is calculated via the processor 141 according to the electrocardiogram. In another embodiment, the physiological signal is a brain wave, the digital signal is an electroencephalogram, and the user status information is a sleep index that is calculated via the processor 141 according to the electroencephalogram. The ExpressCard socket 146 may be electrically connected to the ExpressCard connector 126 of the physiological signal receiving device 120 in order to provide electric power for the battery and power management system 127 of the physiological signal receiving device 120. Therefore, the computing device 140 may serve as a charging device. In some embodiments, the digital signal can be transmitted from the physiological signal receiving device 120 to the processor 141 of the computing device 140 through the ExpressCard socket 146.

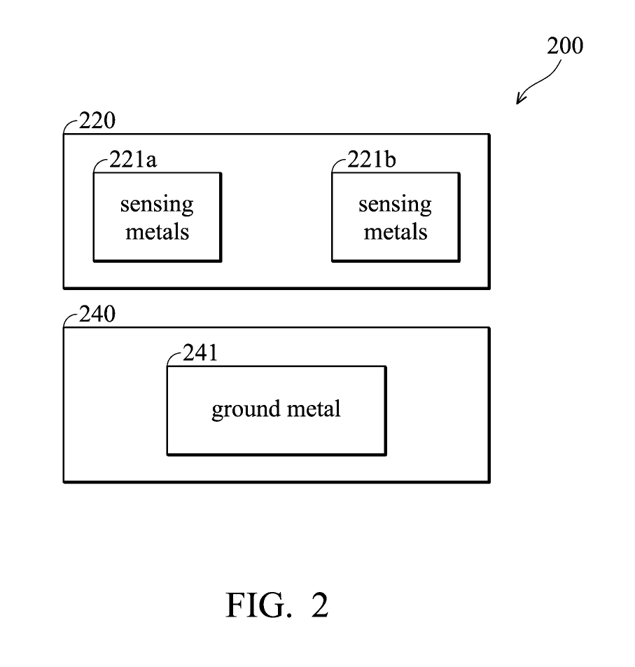

FIG. 2 is an outlook drawing 200 illustrating a physiological signal receiving device according to an embodiment of the invention. A plurality of sensing metals, such as sensing metals 221a and 221b, serve as the sensor 122, disposed on the obverse side 220 of the physiological signal receiving device. Although there are only two sensing metals in FIG. 2, the sensor 122 can comprise more than 2 sensing metals, such as 3 or 4 sensing metals. The physiological signal receiving device 120 can receive a physiological signal from a user, such as heartbeat, pulse, brain waves, body temperature and body fat, through the sensing metals 221a and 221b. There is a ground metal 241 disposed on the back side 240 of the physiological signal receiving device. In some embodiments, the ground metal 241 is optional and can be removed from the back side 240 of the physiological signal receiving device.

In one embodiment of the invention, the physiological signal receiving device 120 is utilized for measuring an electrocardiogram of a user and calculating a sentiment index of the user. The physiological signal receiving device 120 can be applied to a checkup or a polygraph. First, the user puts two thumbs on the sensing metals 221a, 221b of the obverse side 220 of the physiological signal receiving device, respectively. If necessary, other fingers can be put on the ground metal 241 of the back side 240 of the physiological signal receiving device. By measuring potential differences between the sensing metals 221a, 221b, the processor 121 of the physiological signal receiving device 120 can convert a physiological signal (heartbeat or pulse) into a digital signal (electrocardiogram). Next, the physiological signal receiving device 120 wirelessly transmits the electrocardiogram to the processor 141 of the computing device 140. The processor 141 displays the electrocardiogram on the display unit 142, and calculates user status information (sentiment index) according to heart rate and heartbeat waveforms of the electrocardiogram. Then, the processor 141 displays the user status information on the display unit 142. For example, the sentiment index can be ordered from 0 to 100. If the sentiment index is smaller than 40, it means that the user is relax, if the sentiment index is between 40 and 60, it means that the user is not that relaxed buy not tense, if the sentiment index is larger than 60, it means that the user is tense. By calculating the sentiment index, emotions of a user can be quantified, such that, for example, whether or not someone being asked questions is lying may be determined.

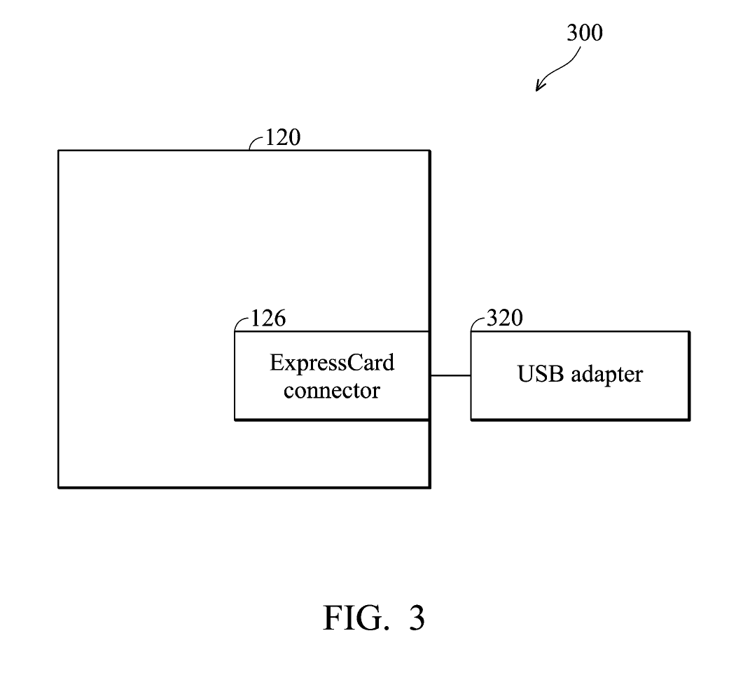

FIG. 3 is a diagram 300 illustrating a physiological signal receiving device adapted to a USB connector according to an embodiment of the invention. For some computers without ExpressCard sockets, the ExpressCard connector 126 may be coupled to a USB socket of a computer through a USB adapter 320. In another embodiment of the invention, the physiological signal receiving device 120 may comprise a Micro USB socket that is directly connected to a USB socket of a computer through a cable in order to charge or transmit a digital signal.

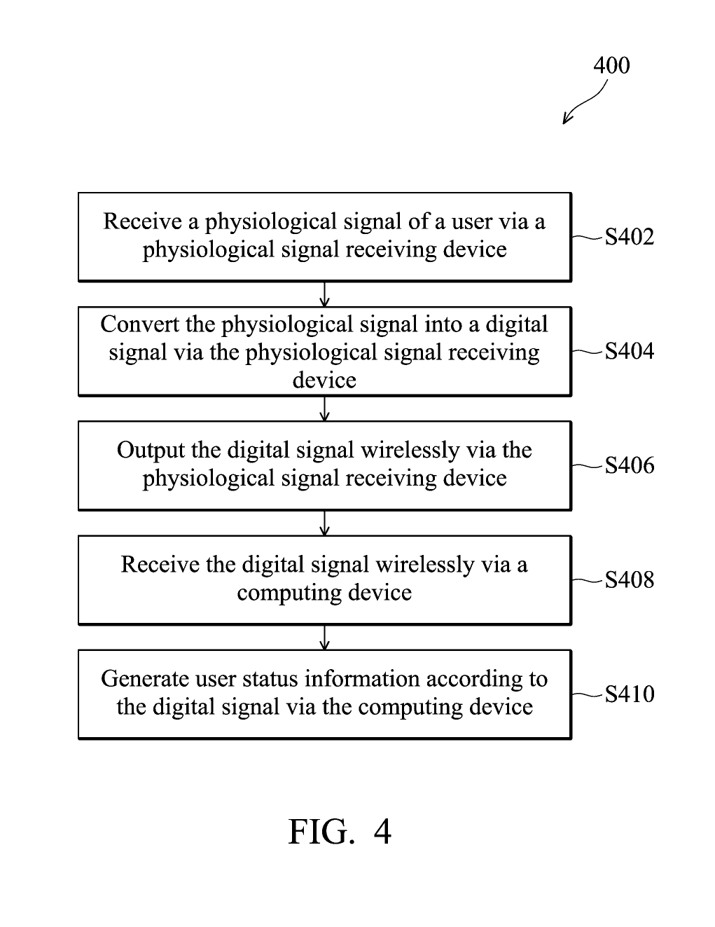

FIG. 4 is a flow chart 400 illustrating a physiological signal receiving method according to an embodiment of the invention. To begin, in step S402, a physiological signal from a user is received via a physiological signal receiving device. The physiological signal may be a heartbeat, pulse, or brain waves. Next, in step S404, the physiological signal is converted into a digital signal via the physiological signal receiving device. The digital signal may be an electrocardiogram or an electroencephalogram. In step S406, the digital signal is wirelessly output via the physiological signal receiving device. In step S408, the digital signal is wirelessly received via a computing device. Finally, in step S410, user status information is generated according to the digital signal via the computing device. The user status information may be a sentiment index or a sleep index. The physiological signal receiving device comprises an ExpressCard interface.

The invention utilizes an ExpressCard as an interface of the physiological signal receiving device because the ExpressCard is cheap, and easy to be incorporated into a notebook, and portable electronic device. Thus, the physiological signal receiving device of the invention has a commercial advantage, wherein people or hospitals may use the device to understand physical conditions of users.

It will be apparent to those skilled in the art that various modifications and variations can be made in the invention. It is intended that the standard and examples be considered as exemplary only, with a true scope of the disclosed embodiments being indicated by the following claims and their equivalents.

What is claimed is:

1. An electronic device for receiving a physiological signal of a user, comprising:
a physiological signal receiving device, comprising:
a sensor, configured to receive the physiological signal of the user;
a first processor, coupled to the sensor, and configured to convert the physiological signal into a digital signal;
a first communication interface, configured to output the digital signal;
a battery and power management system; and
an ExpressCard connector, coupled to the first processor; and
a computing device, comprising:
a second communication interface, configured to receive the digital signal; and
an ExpressCard socket.

2. The electronic device as claimed in claim 1, wherein the computing device further comprises:
a second processor, coupled to the second communication interface, and generating user status information according to the digital signal.

3. The electronic device as claimed in claim 1, wherein the sensor comprises a plurality of sensing metals configured to receive the physiological signal of the user.

4. The electronic device as claimed in claim 1, wherein the ExpressCard connector of the physiological signal receiving device is coupled to the computing device through a USB adapter.

5. The electronic device as claimed in claim 1, wherein the ExpressCard socket is coupled to the ExpressCard connector in order to provide electric power for the battery and power management system of the physiological signal receiving device.

6. The electronic device as claimed in claim 1, wherein the physiological signal is a heartbeat signal, and the digital signal is an electrocardiogram.

7. The electronic device as claimed in claim 1, wherein each of the first and second communication interfaces is a wireless communication module.

8. The electronic device as claimed in claim 1, wherein each of the first and second communication interfaces is a USB interface.

9. The electronic device as claimed in claim 2, wherein the user status information is a sentiment index.

10. A physiological signal receiving device for receiving a physiological signal of a user, comprising:
a sensor, configured to receive the physiological signal of the user;
a processor, coupled to the sensor, and configured to convert the physiological signal into a digital signal;
a communication interface, configured to output the digital signal;
a battery and power management system; and
an ExpressCard connector, coupled to the processor.

11. The physiological signal receiving device as claimed in claim 10, wherein the sensor comprises a plurality of sensing metals configured to receive the physiological signal of the user.

12. The physiological signal receiving device as claimed in claim 10, wherein the ExpressCard connector is coupled to a USB adapter.

13. The physiological signal receiving device as claimed in claim 10, wherein the ExpressCard connector is coupled to a charging device.

14. The physiological signal receiving device as claimed in claim 10, wherein the physiological signal is a heartbeat signal, and the digital signal is an electrocardiogram.

15. The physiological signal receiving device as claimed in claim 10, wherein the communication interface is a wireless communication module.

16. The physiological signal receiving device as claimed in claim 10, wherein the communication interface is a USB interface.

17. A physiological signal receiving method for receiving a physiological signal of a user, comprising:
receiving the physiological signal of the user via a physiological signal receiving device;
converting the physiological signal into a digital signal via the physiological signal receiving device;
outputting the digital signal wirelessly via the physiological signal receiving device;
receiving the digital signal wirelessly via a computing device; and
generating user status information according to the digital signal via the computing device,
wherein the physiological signal receiving device comprises an ExpressCard interface.

18. The physiological signal receiving method as claimed in claim 17, wherein the physiological signal is a heartbeat signal, and the digital signal is an electrocardiogram.

19. The physiological signal receiving method as claimed in claim 17, wherein the user status information is a sentiment index.

20. A computing device for computing a digital signal, comprising:
a processor, generating user status information according to the digital signal.
